(19) United States
(12) Patent Application Publication
(10) Pub. No.: US 2025/0231179 A1
(43) Pub. Date: Jul. 17, 2025

(54) METHODS FOR DETECTING INDOLE, AN INDOLE PRODUCING BACTERIUM, OR AN INDOLE PRODUCING CHEMICAL

(71) Applicants: THE REGENTS OF THE UNIVERSITY OF MICHIGAN, Ann Arbor, MI (US); VIRGINIA COMMONWEALTH UNIVERSITY, Richmond, VA (US)

(72) Inventors: Xuewei Wang, Richmond, VA (US); Mark E. Meyerhoff, Ann Arbor, MI (US); Yuqing Chen, Superior Township, MI (US); Chuanwu Xi, Ann Arbor, MI (US); Tiefei Dong, Ann Arbor, MI (US); Jianfeng Wu, Canton, MI (US); Brock Andrew Brady, Richmond, VA (US); Wuwei Li, Richmond, VA (US)

(21) Appl. No.: 18/854,536

(22) PCT Filed: Apr. 13, 2023
(86) PCT No.: PCT/US2023/018472

§ 371 Date: Oct. 6, 2024

Related U.S. Application Data
(60) Provisional application No. 63/331,054, filed on Apr. 14, 2022.

(51) Int. Cl. G01N 33/52; G01N 21/29; G01N 33/14

(52) U.S. Cl. CPC G01N 33/52; G01N 21/29; G01N 33/14

(57) ABSTRACT

In an example method for detecting indole, an indole producing bacterium, or an indole producing chemical, a polymeric detection tool is exposed to a sample and the polymeric detection tool is monitored for a red color change. The polymeric detection tool includes an acidic and oxygen soluble polymer and a nitrosation agent impregnated in walls of the acidic and oxygen soluble polymer. This example method can be used for detecting an indole producing bacterium in a body fluid sample.

### CROSS-REFERENCE TO RELATED APPLICATIONS

This application claims the benefit of U.S. provisional application Ser. No. 63/331,054, filed Apr. 14, 2022, the contents of which is incorporated by reference herein in its entirety.

### STATEMENT REGARDING FEDERALLY SPONSORED RESEARCH OR DEVELOPMENT

This invention was made with government support under Grant No. R01-EB-023294 awarded by the National Institutes of Health (NIH). The government has certain rights in the invention.

### BACKGROUND

Various sensors have been used in the healthcare field for real-time and continuous analysis of a patient. Some sensors, often referred to as wearables, are positioned on the human body, and enable the detection of various biomarkers, such as heart rate and blood pressure, and/or fluids, such as sweat, tears, saliva, etc. Other sensors are implantable or ingestible, and interface with fluids, such as urine, blood, interstitial fluid, and gastrointestinal fluid, in vivo. As examples, skin-worn sensors, transdermal microneedle patches, tooth-mounted sensors, face mask mounted sensors, swallowable capsule systems, and intravascular and other subcutaneous sensors have been developed to track a wide variety of analytes, such as pH, metabolites, electrolytes, enzymes, macronutrients, pathogens, and drugs.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1 is a chemical scheme depicting the nitrosation reaction of indole (1) by nitrous acid (HNO₂) under acidic conditions;

FIG. 2A is a schematic, perspective, and cut-away view of an example of a polymeric detection tool in the form of a catheter;

FIG. 2B is a schematic and perspective view of an example of a polymeric detection tool in the form of a planar patch;

FIG. 3A is a graph depicting absorbance (arbitrary units, Y-axis) versus wavelength (nm, X-axis) for acidic (180 mM sulfuric acid) nitrite (1 mM) solutions doped with different concentrations of indole;

FIG. 3B is a calibration curve depicting the absorbance at 537 nm (arbitrary units, Y-axis) versus the indole concentration (μM, X-axis) for the indole doped acidic nitrite solutions of FIG. 3A;

FIG. 3C is a graph depicting absorbance at 537 nm (arbitrary units, Y-axis) versus time (minutes, X-axis) for an acidic nitrite (1 mM) solution doped with 100 μM indole;

FIG. 3D is a graph depicting the mass spectrum of reaction products of a reaction between nitrite and indole;

FIG. 4A is a graph depicting absorbance (arbitrary units, Y-axis) versus wavelength (nm, X-axis) for acidic (180 mM sulfuric acid) S-nitroso-N-acetylpenicillamine (SNAP, 1 mM) solutions doped with different concentrations of indole;

FIG. 4B is a calibration curve depicting the absorbance at 537 nm (arbitrary units, Y-axis) versus the indole concentration (μM, X-axis) for the indole doped acidic SNAP solutions of FIG. 4A;

FIG. 4C is a graph depicting absorbance at 537 nm (arbitrary units, Y-axis) versus time (minutes, X-axis) for an acidic SNAP (1 mM) solution doped with 100 μM indole;

FIG. 4D is a bar graph depicting the UV-visible absorbance based selectivity at 537 nm in the presence of common interference species and indole analogs (each at 100 μM);

FIG. 5 is a graph depicting absorbance (arbitrary units, Y-axis) versus wavelength (nm, X-axis) for S-nitroso-N-acetylpenicillamine (SNAP, 1 mM) solutions doped with 0.1 mM indole and varying concentrations of sulfuric acid; and

FIG. 6 is a graph depicting the hue (degrees, Y-axis) versus indole concentration (μM, X-axis) for SNAP impregnated silicone sheets after incubation in indole solutions of varying concentration.

### DETAILED DESCRIPTION

The methods and tools disclosed herein enable the detection of indole, a bacterium that produces indole, or a chemical that produces indole. As such, the indole present in the sample being tested may be naturally present in the sample, or may be present as the result of bacteria, protein, or chemical degradation. Some examples of the methods and tools are suitable for use in vitro, and other examples of the methods and tools are suitable for use in vivo. The in vitro tools and methods may be used for a variety of applications, including, for example, experimental microbiology, bacteria (e.g., Escherichia coli (E. coli)) or chemical (e.g., L-abrine) detection, and assessment of food (e.g., seafood) freshness and/or contamination. The in vivo tool is a dual function device because it functions as both a device (e.g., a catheter) and a sensor for indole, a bacterium that produces indole, or a chemical that produces indole.

### Indole

Indole has the following structure:

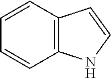

and is produced by eighty five (85) different Gram-positive and Gram-negative bacteria species and from some amino acid derivatives, such as L-abrine. For some of these species, indole is a characteristic metabolite that may be used to indirectly identify the species. Examples of bacteria that are capable of producing indole include E. coli, Klebsiella oxytoca, Proteus vulgaris, Morgenella morganii, Vibrio cholera, Providencia species, Citrobacter koseri, Aeromonas species, Plesiomonas species, Pasteurella species, Cardiobacterium hominis, and Propionibacterium acenes. The indole in these species may be generated by the deamination and hydrolysis of the amino acid tryptophan. Among these bacteria species, E. coli is prevalent in urinary tract infections and may be present in food and water. The tryptophanase in E. coli catalyzes the deamination reaction to produce the indole. L-abrine may be present in food and beverages. N-methyltryptophan oxidase (MTOX) catalyzes the oxidative deamination of L-abrine to generate L-tryptophan, and tryptophanase catalyzes the reversible hydrolytic cleavage of L-tryptophan to produce the indole.

In the examples disclosed herein, the tool that is used includes a nitrosation agent that can convert indole into a nitroso derivative, which undergoes tautomerization and additional reactions (e.g., dimerization and deoximation), generating one or more red products.

### Solution-Based Sensing Methods

Some of the examples disclosed herein utilize the nitrosation agent in solution to detect an indole producing bacteria or an indole producing chemical in a biological, food, or beverage sample. The method includes exposing the biological, food, or beverage sample to a solution of the nitrosation agent, and monitoring the biological, food, or beverage sample for a red color generation.

In the solution-based example, the nitrosation agent may be a nitrite. Examples of suitable nitrites including nitrite salts and organic nitrites. Examples of suitable nitrite salts include alkali metal and alkaline earth metal nitrite salts. Specific examples include nitrite salts of Li, Na, K, Rb, Ca, and Mg. Organic nitrites may also be used, such as ethyl nitrite and amyl nitrite.

The nitrite is dissolved into an acidic solution, because at highly acidic conditions (e.g., 3.0 or less), the nitrite can be converted to nitrous acid (HNO₂, pKₐ ˜3.2), which induces the red color-generating reactions shown in FIG. 1. In FIG. 1, the nitrous acid attacks C-3 of the indole (1) nucleus to generate 3-nitroso indole (2), which tautomerizes to 3-oxime indole (3). Another indole molecule (e.g., present in the food or beverage sample) reacts with 3-oxime indole via 1,2-nucleophilic addition and generates indole red (2-indolyl-3-oxime-3H-indole) (4). Indole red then undergoes deoximation under acidic conditions to form indoxyl red (2-indolyl-3-one-3H-indole) (5). Indoxyl red (2-indolyl-3-one-3H-indole) is an indole compound with a strong purple-red color (exhibiting absorbance peaks at 537 nm).

The nitrite solution may include water or a combination of water and an organic solvent. In one example, the nitrite solution is an aqueous solution that includes water (e.g., deionized water) without any organic solvent(s). In another example, the nitrite solution includes a mixture of water and an organic solvent. Any organic solvent that is miscible or immiscible with an aqueous solution of the nitrite may be used. Examples of the organic solvent include butanol, methanol, tetrahydrofuran, benzene, hexane, and combinations thereof.

In any example of the nitrite solution disclosed herein, the acid that is included to render it acidic is selected from the group consisting of sulfuric acid (H₂SO₄) and hydrochloric acid (HCl).

In one example to generate the solution of the nitrosation agent, the nitrite is dissolved in water, and then the aqueous nitrite solution is mixed with the acid and the organic solvent (if it is included). In another example to generate the solution of the nitrosation agent, water and the acid are pre-mixed, and then the nitrite is dissolved in the water and acid mixture. In still another example to generate the solution of the nitrosation agent, the nitrite is dissolved in the organic solvent to generate a non-aqueous nitrite solution, and then the non-aqueous nitrite solution is mixed with water and the acid.

The concentration of the nitrite in the final acidic solution will depend upon the desired amount of nitrous acid that is to be generated. In an example, the concentration of the nitrite in the acidic solution ranges from about 0.01 mM to about 100 mM. When the nitrite is first dissolved in water to generate an aqueous stock solution, the amount of the aqueous stock solution added to the acid or the combination of the acid and the organic solvent will depend upon the nitrite concentration in the aqueous stock solution and the desired nitrite concentration in the acidic solution.

The concentration of the acid in the final acidic solution will depend upon the desired pH of the acidic solution. In an example, the pH ranges from about 1 to about 3, and the concentration of the acid in the acidic solution ranges from about 1 mM to about 0.2 M.

In one specific example, the solution of the nitrosation agent includes about 1 mM sodium nitrite and about 180 mM sulfuric acid in butanol. This example also includes some water from an aqueous stock solution of the nitrite salt.

As mentioned, the solution of the nitrosation agent may be used to detect indole or an indole producing bacterium or an indole producing chemical in a food (e.g., shrimp) or beverage sample. The method involves exposing the food or beverage sample to the solution of the nitrosation agent, and monitoring the food or beverage sample for a red color change. In one example, the food or beverage may be allowed to incubate in the solution of the nitrosation agent for at least 0.5 hours. If a red color change is observed, the red color is indicative of the presence of indole, and, in some instances, of the bacteria or chemical that generates indole in the presence of acid and oxygen. It is to be understood that the intensity of the color may vary depending upon the amount of indole present.

### Polymeric Detection Tool and Methods

Other examples disclosed herein utilize a polymeric detection tool, which contains the nitrosation agent impregnated in a polymer, to detect indole, an indole producing bacterium or an indole producing chemical in a sample. The method includes exposing the polymeric detection tool to the sample, the polymeric detection tool including an acidic and oxygen soluble polymer and a nitrosation agent impregnated in walls of the acidic and oxygen soluble polymer; and monitoring the polymeric detection tool for a red color change.

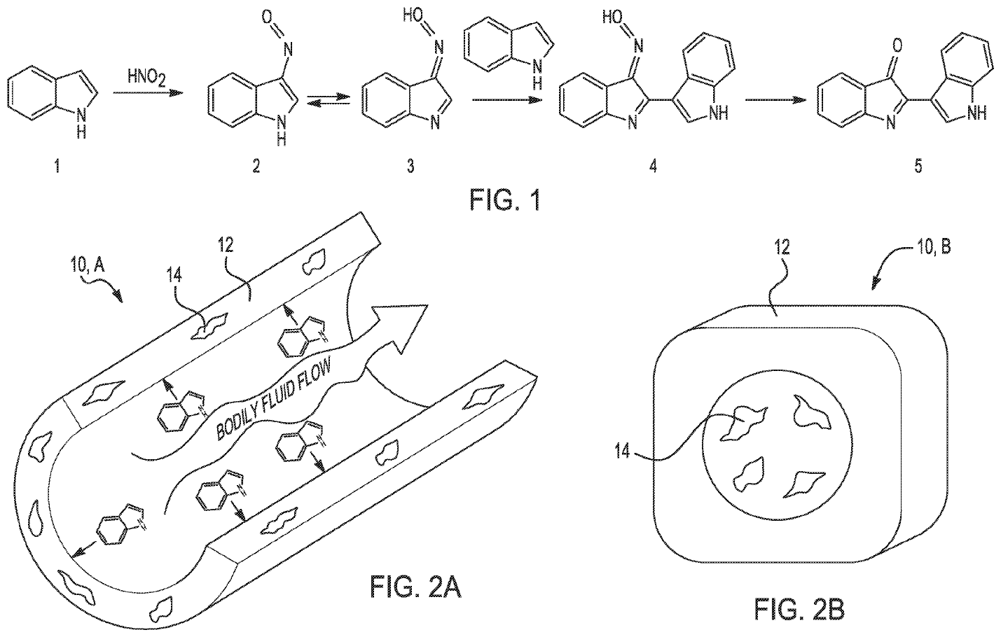

Examples of the polymeric detection tool 10 are shown in FIG. 2A and FIG. 2B. One example of the polymeric detection tool 10, A is shown in FIG. 2A, and this example is catheter or other polymer tube through which the sample can flow. Another example of the polymeric detection tool 10, B is shown in FIG. 2B, and this example is a planar patch.

The polymeric detection tool 10, A, B includes the acidic and oxygen soluble polymer 12. The phrase “acidic and oxygen soluble” means that the polymer has an acidic pH and that oxygen is soluble in the polymer. Several polymers may be considered to be acidic and to exhibit oxygen solubility, but to varying degrees. In the examples disclosed herein, the acidity of the polymer 12 and the oxygen solubility of the polymer 12 should both be sufficient to provide the desired conditions for the chromogenic assay. In an example, the acidity of the polymer 12 ranges from about 0 to about 4. In an example, the oxygen solubility of the polymer 12 ranges from about 0.01 cm³ (STP)/(cm³ polymer) to about 1000 cm³ (STP)/(cm³ polymer).

The acidic and oxygen soluble polymer 12 is capable of incorporating the nitrosation agent 14 into its walls via a solvent impregnation method. Some specific examples of suitable polymers 12 include silicone rubber, polyurethane, a silicone-urethane copolymer, plasticized polyvinyl chloride, polyvinyl alcohol, and polyvinylpyrrolidone. ELAST-EON™ E2As is an example of a siloxane-based polyurethane elastomer commercially available from Aortech Biomaterials, Scoresby Victoria, Australia, and CARBOSIL® is an example of a thermoplastic silicone-polycarbonate-urethane commercially available from DSM Biomedical Inc., Berkeley, CA.

In the polymeric detection tool 10, A, B, the nitrosation agent 14 is impregnated in the walls of the acidic and oxygen soluble polymer 12. Examples of nitrosation agents 14 that are capable of being impregnated into the walls of the polymer 12 include a diazeniumdiolate, a metal-nitrosyl complex, or an S-nitrosothiol. Examples of suitable diazeniumdiolates include dialkylhexamethylenediamine diazeniumdiolates of the form RN[N(O)NO]—(CH₂)₆NH₂⁺R:

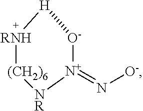

where R is CH₃, CH₂CH₃, (CH₂)₂CH₃, (CH₂)₃CH₃, (CH₂)₄CH₃, (CH₂)₅CH₃, and (CH₂)₁₁CH₃. An example of a suitable metal nitrosyl complex includes sodium nitroprusside. Examples of suitable S-nitrosothiols are selected from the group consisting of S-nitrosoglutathione (GSNO), S-nitroso-N-acetylpenicillamine (SNAP), and S-nitrosocysteine (CysNO).

Each of the nitrosation agents 14 is capable of generating nitric oxide (NO) when exposed to light, heat, pH, metal ions, or moisture (e.g., from a fluid exposed to the polymeric detection tool 10, A, B). For example, SNAP slowly releases NO via hemolytic fission of the S—N bond at body temperature and in the presence of urine, blood, etc. Under aerobic conditions, the NO generates nitrous acid (HNO₂) according to the following reaction mechanisms:

2NO+O₂→2NO₂ (1)

NO₂+NO\vec{←}N₂O₃ (2)

N₂O₃+H₂O→2HNO₂ (3)

As described in reference to FIG. 1, the nitrous acid induces the red color-generating nitrosation reactions with indole. Indole from the sample can be extracted into the polymeric detection tool 10, A, B due to its hydrophobicity, and the high acidity and oxygen solubility enhances the colorimetric reaction with the polymeric matrix.

The nitrosation agent 14 may be incorporated into the walls of the acidic and oxygen soluble polymer 12 via a solvent impregnation method. In this method, the nitrosation agent 14 (which is in solid form) is dissolved in a suitable solvent, such as acetone, ethyl acetate, cyclohexane, isopropanol, methanol, butanone, tetrahydrofuran, etc. It is to be understood that the solvent is also a suitable swelling agent for the acidic and oxygen soluble polymer 12. Once a solution of the nitrosation agent 14 is prepared, the acidic and oxygen soluble polymer 12 is soaked in the nitrosation agent solution. Soaking may be performed for a time sufficient to achieve impregnation of the nitrosation agent 14. In an example, soaking may be performed for up to 72 hours (3 days). In one example, soaking is performed for a time ranging from about 1 min to about 24 hours). After soaking, the acidic and oxygen soluble polymer 12 (now having the nitrosation agent 14 therein) may be rinsed with the solvent and dried (solvent evaporated). This forms the polymeric detection tool 10, A, B.

The concentration of the nitrosation agent 14 may be selected to exceed its solubility in the selected acidic and oxygen soluble polymer 12. At this concentration, a polymer-crystal composite may form during the solvent evaporation portion of the impregnation method. Thus, a solid form of the nitrosation agent 14 is embedded in the walls of the acidic and oxygen soluble polymer 12.

As mentioned, the polymeric detection tool 10, A, B may be used to detect indole or an indole producing bacterium or an indole producing chemical in a biological, food (e.g., shrimp) or beverage sample, such as a bodily fluid, a food, or a beverage. Given the breadth of sample types, the polymeric detection tool 10, A, B may take on many forms; some of which are suitable for use in vivo and others of which are suitable for use ex vivo.

One specific example of an in vivo polymeric detection tool 10, A is shown in FIG. 2A. This polymeric detection tool 10, A is a catheter. Catheters are medical devices that can be inserted into the human body, for example, via a body cavity, duct, or vessel. Catheters may be used in a variety of medical applications because they serve a broad range of functions. In the examples disclosed herein, the catheter/polymeric detection tool 10, A is a dual function device used for fluid drainage and indole sensing.

In the example shown in FIG. 2A, the acidic and oxygen soluble polymer 12 is in the form of a catheter tube, and the nitrosation agent 14 is contained within its walls. The acidic and oxygen soluble polymer 12 prevents the nitrosation agent 14 from rapid leaking out of the walls, but is permeable to moisture from the sample running through the central portion of the catheter tube or from a thin water layer surrounding the outside of the catheter tube. When exposed to moisture or body temperature, the nitrosation agent 14 degrades to form nitric oxide, which generates nitrous acid (HNO₂) (see mechanisms 1-3). When indole, an indole producing bacterium, or an indole producing chemical is present in the sample being drained with the catheter, the nitrous acid nitrosates indole at the C-3 position of the chemical structure to generate indoxyl red. Indoxyl red changes the color of the polymer 12, and thus indicates the presence of the indole, the indole producing bacterium, or the indole producing chemical. The color changing reaction can readily occur within the walls of the polymeric detection tool 10, A, allowing for in-situ indication of potential infection(s) in the sample being drained from the patient.

A tube shaped polymeric detection tool 10, A may also be used in in vitro applications, where the tube and sample are added to a petri dish or other suitable vessel for testing the sample for indole, indole producing bacterium, or

One specific example of an in vitro polymeric detection tool 10, B is shown in FIG. 2B. This polymeric detection tool 10, B is a planar patch. Planar patches are medical devices that can be affixed to the human body, e.g., to cover a wound. The planar patch/polymeric detection tool 10, B is a dual function device used for wound coverage and indole sensing.

In the planar patch, at least a portion of the planar patch is a sensor made up of the acidic and oxygen soluble polymer 12 with the nitrosation agent 14 embedded therein. Whether the acidic and oxygen soluble polymer 12 forms all or a portion of the planar patch, the acidic and oxygen soluble polymer 12 is in the form of a relatively flat cube, relatively flat rectangular box, or other relatively flat three-dimensional shape. The acidic and oxygen soluble polymer 12 does have a length, width, and a depth; however the depth may be relatively small so that the planar patch is flexible and able to conform to the shape of a desired subject (e.g., a limb, appendage, etc. that the planar patch is affixed, adhered, or otherwise secured to). As an example, the “relatively flat” depth means that the z-dimension of the planar patch is minimal (e.g., less than 1 mm).

In the example shown in FIG. 2B, the acidic and oxygen soluble polymer 12 (with the nitrosation agent 14 embedded therein) is constructed so that it is in contact with the surface of the desired subject, while the remainder of the patch is made up of a suitable adhesive or cloth material for securing the planar patch to the subject. In this example, a portion of the planar patch is used for sensing. Alternatively, the entire planar patch may be formed of the acidic and oxygen soluble polymer 12 with the nitrosation agent 14 embedded therein.

Similar to the planar patch shown in FIG. 2B, the acidic and oxygen soluble polymer 12 prevents the nitrosation agent 14 from rapid leaking out of the walls, but is permeable to moisture from a sample at the surface of the subject. When exposed to moisture, the nitrosation agent 14 degrades to form nitric oxide, which generates nitrous acid (HNO₂) (see mechanisms 1-3). When indole, an indole producing bacterium, or an indole producing chemical is present in the sample being drained using the catheter, the nitrous acid nitrosates indole at C-3 and yields indoxyl red, which changes the color of the acidic and oxygen soluble polymer 12 and indicates the presence of the indole, indole producing bacterium, or

The polymeric detection tool 10, A, B may be in the form of other medical devices and sensors.

The method involves exposing the polymeric detection tool 10, A, B to the sample, and monitoring the polymeric detection tool 10, A, B for a red color change. In one example, the polymeric detection tool 10, A, B may be allowed to incubate in the sample for at least 0.5 hours. If a red color change is observed, the red color is indicative of the presence of indole, and, in some instances, of the bacteria or chemical that generates indole in the presence of acid and oxygen. It is to be understood that the intensity of the color may vary depending upon the amount of indole present.

To further illustrate the present disclosure, examples are given herein. It is to be understood that these examples are provided for illustrative purposes and are not to be construed as limiting the scope of the present disclosure.

### EXAMPLES

### Example 1

To test the colorimetric detection of indole based on the nitrosation reaction (see FIG. 1) in solution, nitrite was used.

A stock solution of 200 mM sodium nitrite was prepared in deionized water.

Stock indole solutions were prepared by diluting indole with n-butanol to obtain 100 μM, 75 μM, 50 μM, 30 μM, 10 μM, and 5 μM indole solutions. A control solution of n-butanol was also utilized without any indole (i.e., 0 μM indole).

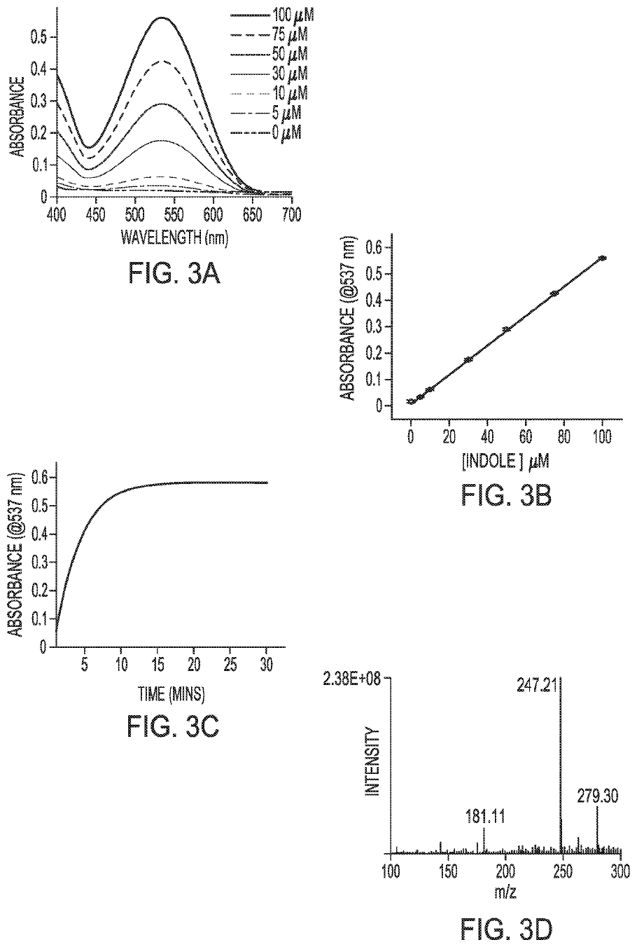

Each of the indole solutions and the control solution was mixed with the nitrite solution, and 180 mM sulfuric acid was added. The respective combined solutions were vortexed for about 10 seconds to mix the reactants. The combined solutions were allowed to incubate at room temperature for about 0.5 hours. The absorbance was then measured using an Agilent 8453 UV-Visible Spectrophotometer with 1.5 mL polystyrene cuvettes. The results are shown in FIG. 3A. As shown in FIG. 3A, the absorbance at 537 nm for the combined solutions increased as the indole concentration increased from 0 M to 100 μM. FIG. 3B illustrates the calibration curve for the results shown in FIG. 3A at absorbance of 537 nm. It is noted that the data points in FIG. 3A and FIG. 3B represent the average±standard deviation for n=3 trials. From the calibration, it was determine that both high linearity (R²=0.999) and a high limit of detection (LOD) (2.94 μM (3σ/s)) were obtained.

Kinetics measurements were performed every 60 seconds with the combined solution containing 1 mM sodium nitrite, 100 M indole, and 180 mM sulfuric acid. FIG. 3C is a graph depicting the absorbance (at 537 nm)-time trace for this combined solution. The kinetics in FIG. 3C illustrate that the reaction was complete within 30 minutes.

A solution containing 1 mM sodium nitrite (from the stock solution), 180 μM sulfuric acid, and 500 μM indole was prepared and allowed to react for 30 minutes. To confirm the products of the reaction, this solution was diluted 100 times with 70:30 methanol: DI water and was tested using a Xevo TQ-S micro Triple Quadrupole Mass Spectrometer (which has a unit mass resolution). The flow rate was 5 mL/minute. The fluidics system was washed and purged between each sample with 50:40:10 acetonitrile:methanol:water. These results are shown in FIG. 3D. As depicted, a dominant peak was observed at m/z=247.21, which correlates with indoxyl red. The peaks at m/z=181.11 and 279.30 are indicative of indole adducts with two methanol groups without sulfuric acid (m/z=181.11) and with sulfuric acid (m/z=279.30).

The nitrite-based nitrosation reaction illustrated in this example represents a novel colorimetric method for detecting indole in a solution phase. This method has comparable if not better analytical performance (LOD, sensitivity, response time, and specificity) than the standard indole indication method based on Kovac's reagent containing a concentrated mineral acid, an alcohol such as isoamyl alcohol or n-butanol, and p-dimethylaminobenzaldehyde that forms a colored adduct with indole.

### Example 2

The sensing capability of S-nitroso-N-acetylpenicillamine (SNAP) was tested in the solution phase.

A stock solution of 200 mM SNAP was prepared in n-butanol. The solid SNAP crystals were green, having light absorption from 530 nm to 630 nm.

Stock indole solutions were prepared by diluting indole with n-butanol to obtain 100 μM, 75 μM, 50 μM, 30 μM, 10 μM, and 5 μM indole solutions. A control solution of n-butanol was also utilized without any indole (i.e., 0 M indole).

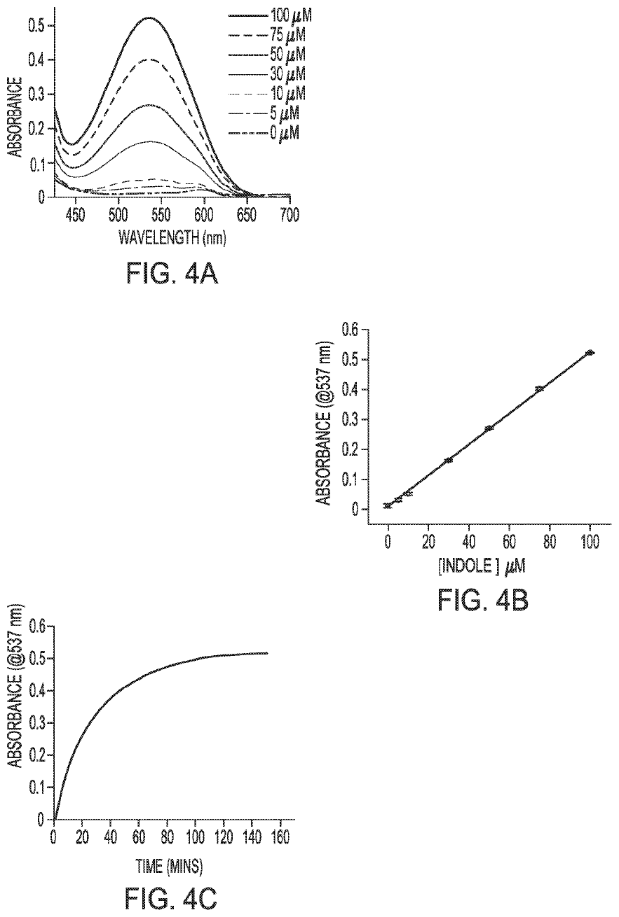

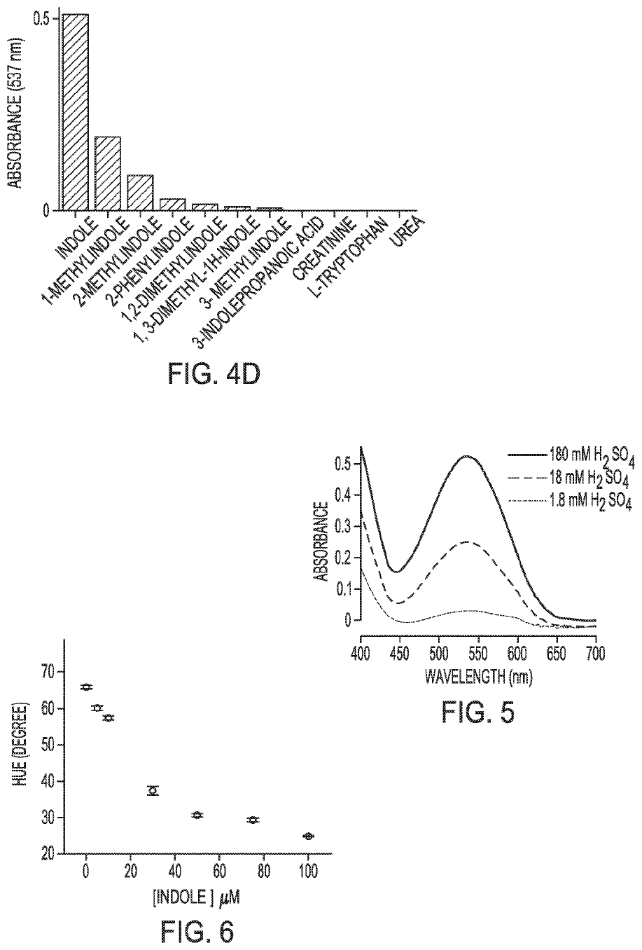

Each of the indole solutions and the control solution was mixed with the SNAP solution, and 180 mM sulfuric acid was added. The respective combined solutions were vortexed for about 10 seconds to mix the reactants. The combined solutions were allowed to incubate at room temperature for about 2.5 hours. The absorbance was then measured using an Agilent 8453 UV-Visible Spectrophotometer with 1.5 mL polystyrene cuvettes. The results are shown in FIG. 4A. In FIG. 4A, a small absorption peak at approximately 600 nm was observed at lower indole concentrations. This was due to unreacted SNAP molecules. Upon reaction with higher concentrations of indole, SNAP absorption disappears, and the new peak at 537 nm was observed. Similar to nitrite, the absorbance at 537 nm for the combined solutions increased as the indole concentration increased. FIG. 4B illustrates the calibration curve for the results shown in FIG. 4A at absorbance of 537 nm. It is noted that the data points in FIG. 4A and FIG. 4B represent the average±standard deviation for n=3 trials. From the calibration, it was determine that both high linearity (R²=0.999) and a high limit of detection (LOD) (5.24 μM (3σ/s)) were obtained.

Kinetics measurements were performed every 60 seconds with the combined solution containing 1 mM SNAP, 100 μM indole, and 180 mM sulfuric acid. FIG. 4C is a graph depicting the absorbance (at 537 nm)-time trace for this combined solution. The kinetics in FIG. 4C illustrate that the reaction was complete within 2.5 hours. Comparing FIG. 3C and FIG. 4C, under the same reaction conditions, SNAP reacted slower with indole than sodium nitrite. As opposed to the immediate availability of HNO₂ in an acidified nitrite solution, the generation of HNO₂ from SNAP involved multiple steps, including SNAP decomposition to form NO and then reaction mechanisms 1-3, which may explain the different reaction kinetics.

Comparative solutions containing 1 mM SNAP, 100 μM indole analogs (1-methylindole and the other indole analogs shown on the X-axis in FIG. 4D) or other urine components (creatinine, L-tryptophan, and urea), and 180 mM sulfuric acid were prepared and analyzed using the UV-Visible Spectrophotometer. The results for the comparative solutions (measured at 537 nm) are plotted in FIG. 4D along with the results for an example solution (labeled “indole”) containing 1 mM SNAP, 100 μM indole, and 180 mM sulfuric acid. The results in FIG. 4D illustrate the selectively of the reaction between SNAP and indole over other indole analogs and other urine components. In particular, it is noted that the other common urine metabolites did not cause any interference. Tryptophan is the substrate of tryptophanase in E. coli to generate indole, and the results in FIG. 4D indicate that tryptophan will not interfere with the SNAP-indole reaction, or the indication of E. coli based on indole. 3-methylindole, 3-indolepropionic acid, and tryptophan may have negligible responses due to their substituted C-3. Although other indole analogs, such as 1-methylindole and 2-methylindole, show absorbance-based responses (likely due to the available C-3), they do not exist in urine and therefore would not interfere with the in-situ detection of E. coli-generated indole in the urinary tract.

Oxygen is needed for the reaction of SNAP and indole according to reaction mechanisms 1-3. To validate that O₂ was needed, the SNAP-indole reactions were carried out with oxygenated and deoxygenated solutions. For this evaluation, two solutions were prepared with 1 mM SNAP and 180 mM H₂SO₄ in n-butanol. Nitrogen gas was purged deep into one solution with a single exit port to regulate the pressure. Another solution was prepared similarly with oxygen gas. The respective gasses were purged into the solutions for 90 s/mL to allow for gas exchange. A 100 μL solution with a 10 times higher indole concentration was injected into each solution (total indole=0.1 mM) to initiate the SNAP-indole reactions. This concentrated indole stock solution was used to minimize excess O₂ being introduced into the sample. Each solution was allowed to react for 1 hour under constant gas influx.

### Example 3

The color of each solution was monitored and photographs were taken at the following intervals after indole introduction: 5 minutes, 10 minutes, 20 minutes, 30 minutes, 45 minutes, and 60 minutes. While the photographs are not reproduced herein, the reaction proceeded much faster in the presence of oxygen. A very light pink color was observed 5 minutes after indole introduction for the solution purged with oxygen, and the color intensified as time progressed. In contrast, no color was observed for the solution purged with nitrogen until 45 minutes after indole introduction, at which time the very light pink color was observed. These results confirmed the role of oxygen in the SNAP-indole reaction.

### Example 4

To determine the effect of acidity on the SNAP-indole reactions, these reactions were carried out with different concentrations of sulfuric acid. For this evaluation, three solutions were prepared with 1 mM SNAP in n-butanol, with 0.8 mM H₂SO₄, 18 mM H₂SO₄, and 180 mM H₂SO₄, respectively. The solutions were allowed to incubate at room temperature for about 2.5 hours. The absorbance was then measured using an Agilent 8453 UV-Visible Spectrophotometer with 1.5 mL polystyrene cuvettes. The results are shown in FIG. 5. As depicted, lower concentrations of sulfuric acid significantly reduced the generation of the red product. These results confirmed that a highly acidic pH accelerated the decomposition of SNAP and ensured the undissociated form of nitrous acid for nitrosation, rendering the SNAP-indole reaction pH-dependent.

### Example 5

In this example, SNAP was embedded in the walls of silicone rubber tubing and sheets using a solvent impregnation method as described herein.

Tetrahydrofuran was used as the solvent for SNAP and the swelling agent for the silicone rubber. Approximately 1-cm sections of Bardex Foley catheter and ¼ inch circles of 0.3-μm thick silicone rubber sheet were placed in a 100 mg/mL SNAP solution for 10 minutes in the dark. The swollen sheets and catheter segments were removed from the tetrahydrofuran solution and allowed to dry for 12 hours, which allowed the tetrahydrofuran to evaporate. After evaporation was complete, the SNAP impregnated catheters and sheets had returned to their original size. The SNAP impregnated catheters and sheets were green, due to the inclusion of the green SNAP crystals.

The SNAP impregnated catheters and sheets were washed with ethanol to remove excess SNAP from the surfaces.

Some of the SNAP impregnated sheets were placed into solutions containing indole for 3 hours (at 37° C.). Bis-Tris buffer at pH 6.5 was used to mimic the average pH of urine. The SNAP impregnated sheets were removed from the respective solutions and dabbed dry with a KIMWIPER (Kimberly-Clark). Photos were then obtained using a 3D-printed dark box equipped with an iPhone SE. The flashlight was used as the sole light source. The position of each SNAP impregnated sheet fixed for photography. Hue values were extracted using the Color Mate app. The quantitative hue values are shown in FIG. 6 (data points represent the average±standard deviation for n=3 trials).

As shown in FIG. 6, indole induced a color shift of the SNAP impregnated sheets toward red (0° C.) as the indole concentration increased, as evidenced by the decreasing hue values. While the solution phase data is not shown, no color was observed, suggesting that indole was extracted into the silicone rubber and reacted with SNAP in the solid matrix.

Fresh human urine samples from a healthy donor were doped with increasing concentrations of indole. Some of the SNAP impregnated silicone sheets and some of the SNAP impregnated silicone catheters were incubated in the doped urine samples at 37° C. for 3 hours. At the 3 hour point, the sheets and catheters were visually inspected for any color change. For both the sheets and the catheters, a colorimetric shift from green to red was observed, indicating that the SNAP impregnated silicone materials work in undiluted urine as well as in buffer. The colors were darker for the SNAP impregnated silicone catheters, indicating that the synthesis process and physicochemical properties of different silicone rubber products can vary. These results indicated that indwelling urinary catheters can readily serve as an on-body colorimetric indole sensor after SNAP impregnation.

A liquid bacterial culture was prepared by inoculating 50 mL of Luria Broth media with a single colony of E. Coli (ATCC 53496). Bacteria were allowed to grow overnight at 37° C. The culture was diluted to 104, 105, and 106 CFU/mL with 10 times diluted sterile tryptone water (5 mg/mL NaCl+1 mg/mL tryptone). Solutions were then transferred to a 24-well plate (1 mL per well).

Some of the SNAP impregnated silicone rubber sheets were sterilized in 70% ethanol for 15 minutes before being placed into the bacterial solutions and incubated at 37° C. Sensors were removed, washed, and photographed after 6 hours. While the photographs are not reproduced herein, the SNAP impregnated silicone rubber sheets exposed to 10⁴ CFU/mL E. coli were green, while the SNAP impregnated silicone rubber sheets exposed to 10⁵ and 10⁶ CFU/mL E. coli experienced an appreciable colorimetric response. Since a lower concentration of E. coli exists in people without infections and 10⁵ CFU/mL coliforms is a threshold for determining an asymptomatic disease state requiring treatment versus the healthy state, this detection range is suitable for the detection/diagnosis of urinary tract infections. The E. coli detection is not as fast as pure indole detection, since the indole needs to be synthesized by the bacterium and then extracted into the silicone rubber matrix to react with the SNAP. However, current urinary tract infection analysis requires sample acquisition and analysis. The data set forth in this example suggests that early diagnosis of urinary tract infections using the naked eye is possible by functionalizing an exposed section of urinary catheters with the proposed molecular probe. Moreover, other common uropathogenic bacterial species, such as P. mirabilis and K. pneumonia, cannot generate indole and cannot generate any red color on an indole sensor (data not shown).

### Comparative Example 6

Kovac's reagent is one standard technique for indole detection. This technique uses p-dimethylaminobenzaldehyde. To compare SNAP with p-dimethylaminobenzaldehyde, the same impregnation method described in Example 6 was used to impregnate p-dimethylaminobenzaldehyde into silicone rubber. Even at 100 M of doped indole, the silicone rubber experienced no color change even after 24 hours of incubation at 37° C.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range. For example, a range from about 0 to about 4 should be interpreted to include not only the explicitly recited limits of 0 to about 4, but also to include individual values, such as 1, 2.3, 3.5, etc., and sub-ranges, such as from about 1.5 to about 3, from about 2 to about 4, etc. Furthermore, when “about” is utilized to describe a value, this is meant to encompass minor variations (up to +/−10%) from the stated value.

In describing and claiming the examples disclosed herein, the singular forms “a”, “an”, and “the” include plural referents unless the context clearly dictates otherwise.

While several examples have been described in detail, it is to be understood that the disclosed examples may be modified. Therefore, the foregoing description is to be considered non-limiting.

What is claimed is:

1. A method for detecting indole, an indole producing bacterium, or an indole producing chemical, the method comprising:
exposing a polymeric detection tool to a sample, the polymeric detection tool including:
an acidic and oxygen soluble polymer; and
a nitrosation agent impregnated in walls of the acidic and oxygen soluble polymer; and
monitoring the polymeric detection tool for a red color change.

2. The method as defined in claim 1 wherein the acidic and oxygen soluble polymer is selected from the group consisting of silicone rubber, polyurethane, a silicone-urethane copolymer, plasticized polyvinyl chloride, polyvinyl alcohol, and polyvinylpyrrolidone.

3. The method as defined in claim 1 wherein the nitrosation agent is a diazeniumdiolate, a metal-nitrosyl complex, or an S-nitrosothiol.

4. The method as defined in claim 3 wherein the nitrosation agent is the S-nitrosothiol, and wherein the S-nitrosothiol is selected from the group consisting of S-nitrosoglutathione (GSNO), S-nitroso-N-acetylpenicillamine, and S-nitrosocysteine (CysNO).

5. The method as defined in claim 3 wherein the nitrosation agent is the diazeniumdiolate, and the diazeniumdiolate is a dialkylhexamethylenediamine diazeniumdiolate.

6. The method as defined in claim 3 wherein the nitrosation agent is the metal-nitrosyl complex, and the metal-nitrosyl complex is sodium nitroprusside.

7. The method as defined in claim 1, wherein the sample is a bodily fluid, a food, or a beverage.

8. A method for detecting an indole producing bacterium in a body fluid sample, comprising:
exposing an acidic and oxygen soluble polymeric catheter impregnated with a nitrosation agent to a body fluid sample; and
monitoring the acidic and oxygen soluble polymeric catheter for a red color change.

9. The method as defined in claim 8 wherein the acidic and oxygen soluble polymer is selected from the group consisting of silicone rubber, polyurethane, a silicone-urethane copolymer, plasticized polyvinyl chloride, polyvinyl alcohol, and polyvinylpyrrolidone.

10. The method as defined in claim 8 wherein the nitrosation agent is a diazeniumdiolate, a metal-nitrosyl complex, or an S-nitrosothiol.

11. The method as defined in claim 10 wherein the nitrosation agent is the S-nitrosothiol, and wherein the S-nitrosothiol is selected from the group consisting of S-nitrosoglutathione (GSNO), S-nitroso-N-acetylpenicillamine, and S-nitrosocysteine (CysNO).

12. The method as defined in claim 10 wherein the nitrosation agent is the diazeniumdiolate, and the diazeniumdiolate is a dialkylhexamethylenediamine diazeniumdiolate.

13. The method as defined in claim 10 wherein the nitrosation agent is the metal-nitrosyl complex, and the metal-nitrosyl complex is sodium nitroprusside.

14. A method for detecting indole, an indole producing bacterium, or an indole producing chemical, comprising:
exposing a food or beverage sample to a solution of a nitrosation agent; and
monitoring the food or beverage sample for a red color change.

15. The method as defined in claim 14, wherein the nitrosation agent is a nitrite salt dissolved in an acidic solution, and wherein the acidic solution includes butanol and sulfuric acid.
